(19) United States
(12) Patent Application Publication
(10) Pub. No.: US 2003/0096256 A1
(43) Pub. Date: May 22, 2003

(54) Additive to add discrimination means and matter maintaining discrimination information which containing the additive

(75) Inventors: Hisao Itoh, Tokyo (JP); Yukio Itakura, Tokyo (JP); Masaki Hashiyada, Sendai (JP)

Correspondence Address: KODA & ANDROLIA, Suite 3850, 2029 Century Park East, Los Angeles, CA, 90067-3024, US

(73) Assignee: ID TECHNICA CORPORATION

(21) Appl. No.: 10/118,798

(22) Filed: Apr. 9, 2002

(30) Foreign Application Priority Data
Nov. 21, 2001 (JP) 2001-355502

(51) Int. Cl. C12Q 1/68; G06F 19/00; G01N 33/48; G01N 33/50

(52) U.S. Cl. 435/6; 702/20

(57) ABSTRACT

An additive consisting of composition matter made up, according to a discrimination information composed of more than one signs repeatedly appear as more than one patterns, by making each of specific materials be correspond to each of the patterns, is added to a matter to be discriminated to add discrimination information that there is no fear of being stolen by third party. The more than one signs may be 0 and 1, and the more than one patterns may be 00, 01, 10 and 11. Further, the specific materials may be adenine, cytosine, guanine and thymine, and the composition matter may be artificial DNA. The matter maintaining discrimination information may be such matter to be identified directly like commodity or ID cards. Or it may be labels to be attached on objectives to be identified or such matter like ink printed on objectives to be identified.

### BACKGROUND OF THE INVENTION

1. Technical Field of the Invention

This invention related to additive to add discrimination means used for identifying person in security control, verifying whether a matter is true commodity, and so on, and also relates to matter maintaining discrimination information which containing the additive.

2. Prior Art

To identify person or commodity, commonly adopted is such a method where discrimination information is recorded on cards or tags by using magnetism or barcode so as being readable by any way except a vision, and to meet the requirement, the discrimination information is read by certain reader such as a barcode reader and verification is conducted.

However, in recent years, as information technology is widespread, it can be easy to steal an information recorded by way that it can not be read by a vision, and especially forging credit cards becomes a serious social problem. Then, according to the discrimination information recorded by above-described prior way, to prevent an abuse with third party, the problems of most that requires careful attention has occurred on occasion of use.

Accordingly, the present invention aims to provide an additive to add discrimination means that there is no fear of being stolen by third party, and to provide a matter maintaining discrimination information which containing the additive.

### SUMMARY OF THE INVENTION

The additive to add discrimination means, which relates to the present invention, is characterized by consisting of composition matter made up, according to a discrimination information composed of more than one signs repeatedly appear as more than one patterns, by making each of specific materials be correspond to each of the patterns.

The matter maintaining discrimination information, which relates to the present invention, is characterized by containing the additive.

According to the additive to add discrimination means, which relates to the present invention, by analyzing arrangement of material making up composition matter and by applying the patterns of signs to each of the material, it becomes possible for a person who will conduct identification to get discrimination information. Namely, the composition matter itself of the additive becomes the discrimination means. Here, as it is impossible to obtain the discrimination information without depending on analyzing, there is no fear that the information is stolen easily by simple operation such as prior reading operation. Further, as it is impossible to make the discrimination information reappeared without making up composition matter by arranging specific materials (ex; the material stated in the claim 4) according to the discrimination information, there is no fear that the information is forged easily. Moreover, as the discrimination means can be added by only adding , despite appearance of commodity needs to be verified, it is possible to form discrimination matter maintaining discrimination information which can be identified.

Further, the matter maintaining discrimination information, which relates to the present invention, is to maintain the discrimination information indirectly through the discrimination means added by the additive. Then, as the discrimination information is that there is no fear of being stolen or forged easily, highly reliable identification can be achieved.

Preferably, said more than one signs are 0 and 1.

In this case, as the discrimination information becomes digital, it is possible to use common technology of information processing.

Preferably, said more than one patterns are 00, 01, 10 and 11.

In this case, as digitized discrimination information can be replaced by four kinds of materials, structure of the composition matter can be relatively simplified.

Preferably, said specific materials are adenine, cytosine, guanine and thymine, and said composition matter is artificial DNA.

In this case, to obtain the discrimination information, technology of DNA analyzing can be adapted.

Here, the artificial DNA means a DNA base array made up by artificially defining the arrangement of four kinds of base, adenine, cytosine, guanine and thymine, those make up DNA.

Preferably, said discrimination information is an information obtained by encoding combination of numbers of repeating times of short tandem repeat (STR) in DNA base array.

In this case, personal discrimination information with high discrimination accuracy, which differs from each person, can be achieved. Further, as basing on DNA every one has, it can be made by standardized method. Furthermore, as combination of numbers of repeating times of STR is used, and as encoded process is also conducted, leakage of DNA information itself can be prevented, and then privacy can be protected.

Here, encoded information is to include an information processed through a one-way function like hash algorithm.

### BRIEF DESCRIPTION OF THE DRAWINGS

FIG. 1 is a flow chart shows an outline of manufacturing process of the additive in respect of the present invention.

### DETAILED DESCRIPTION OF THE PREFERRED EMBODIMENT

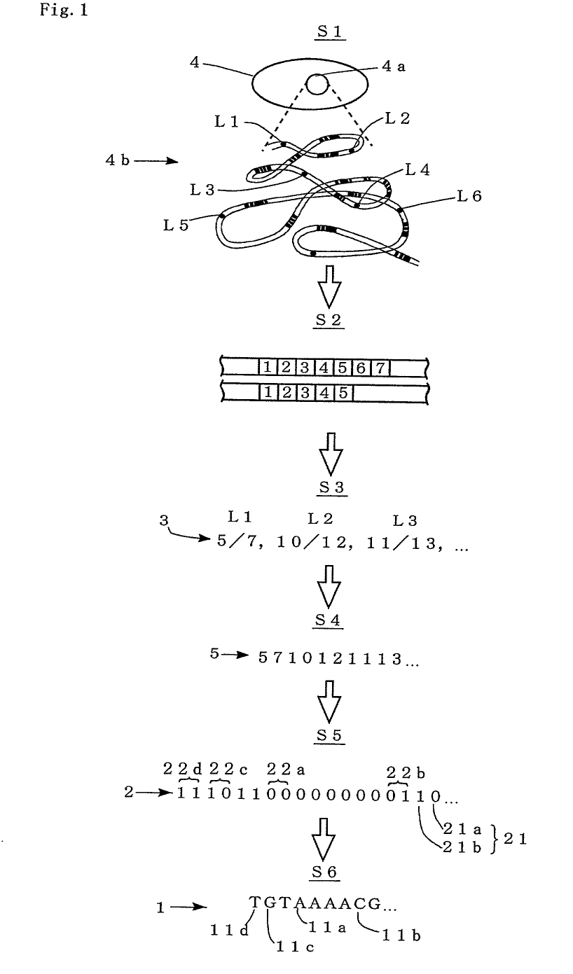

As referring FIG. 1, to be described is an embodiment of additive to add discrimination means in respect of the present invention. FIG. 1 is a flow chart showing an outline of manufacturing process of the additive in respect of the present invention.

The additive consists of composition matter 1. The composition matter 1 is made up, according to a discrimination information 2 composed of more than one signs 21 repeatedly appear as more than one patterns 22, by making each of specific materials 11 be correspond to each of the patterns 22.

According to the additive consists of the composition matter 1, by analyzing arrangement of material 11 making up the composition matter 1 and by applying the patterns 22 of signs to each of the material 11, it becomes possible for a person who will conduct identification to get discrimination information 2. Namely, composition matter 1 itself of the additive becomes the discrimination means. Here, as it is impossible to obtain the discrimination information 2 without depending on analyzing, there is no fear that the information is stolen easily by simple operation such as prior reading operation. Further, as it is impossible to make the discrimination information 2 reappeared without making up the composition matter 1 by arranging specific materials 11 according to the discrimination information 2, there is no fear that the information is forged easily. Moreover, as the discrimination means can be added by only adding, despite appearance of commodity needs to be verified, it is possible to form discrimination matter maintaining discrimination information which can be identified.

Further, the matter maintaining discrimination information is to maintain the discrimination information 2 indirectly through the discrimination means added by the additive. Then, as the discrimination information 2 is that there is no fear of being stolen or forged easily, highly reliable discrimination can be achieved. Here, the matter maintaining discrimination information may be such matter to be identified directly like commodity or ID cards. Or it may be labels to be attached on objectives to be identified or such matter like ink printed on objectives to be identified. In case where the matter maintaining discrimination information is ink, there is such advantages that labels to be attached on objectives to be identified can be mass-produced by presswork or it can be adapted to the any kind of goods where it can be applied.

The more than one signs 21 composing the discrimination information are 0 (indicated as 21a in FIG. 1) and 1 (indicated as 21b in FIG. 1).

To do like this, as the discrimination information 2 becomes digital, it is possible to use common technology of information processing.

The more than one patterns 22 are 00 (indicated as 22a in FIG. 1), 01 (indicated as 22b in FIG. 1), 10(indicated 22c in FIG. 1) and 11 (indicated as 22d in FIG. 1).

To do like this, as digitized discrimination information 2 can be replaced by four kinds of materials, structure of the composition can be simplified relatively.

The specific materials 11 are adenine 11a, cytosine 11b, guanine 11c and thymine 11d. Then the composition matter 1 is artificial DNA.

To do like this, to obtain the discrimination information, technology of DNA analyzing can be adapted.

Next to be described is an embodiment of manufacturing method of the additive to add discrimination means in respect of the present invention.

A manufacturing method described below is adapted to the case where an additive is manufactured by using personal DNA base array.

First of all, in first process S1 (process of extracting DNA), DNA 4b is extracted from cell nucleus 4a in cell 4 of person to be identified. The cell 4, for example, may be got in mouth of the person to be identified.

Next, in process S2 (process of analyzing STR), short tandem repeat (STR) on extracted DNA 4b is analyzed. Base pattern of the analyzing is decided for each of STR seat position, and AATG is the base pattern at example showed in FIG. 1. Analyzing is conducted at several STR seat positions (locus) L1, L2, L3, L4, L5, L6 . . . FIG. 1 shows an example where pattern of AATG appears five times and seven times at STR seat position L1. In FIG. 1 six analyzing positions, from STR seat position L1 to L6 are showed, however, number of analyzing positions is not limited and it can be suitably decided according to discrimination accuracy required for discrimination information.

Result obtained in the process S2 is, in process S3 (process of displaying analyzing result), displayed as combination 3 of numbers of repeating times. The combination 3 of numbers of repeating times showed in FIG. 1 is obtained by putting pairs of numbers of repeating times at STR seat places L1, L2, L3, . . . in order (the pair is called STR alleles). Here, numerical values of STR allele at STR seat position are put in small order like 5,7. So are others at other STR seat positions. Then, in process S4 (process of making DNA personal ID), by basing on the combination 3 of numbers of repeating times, DNA personal ID 5 is made.

The DNA personal ID 5 is encoded in process S5 (encoding process), and binary discrimination information 2 is made. The encoding is conducted for the purpose of ethical consideration, or protecting privacy. Therefore, one-way mapping wherein the DNA personal ID 5 is not be specified from discrimination information 2 is preferable, and process using a hash algorithm, for example, may be conducted.

Continuously, in final process S6 (process of converting base array), according to the discrimination information 2 made in said process S5, material 11 is put so as adenine (11a), cytosine (11b), guanine (11c) and thymine (11d) are correspond to pattern 22a (00), to pattern 22b (01), to pattern 22c (10), and to pattern 22d (11) respectively, and the composition matter 1 is made up, and then the additive is produced.

According to the discrimination information 2 produced by this manufacturing method, personal discrimination information with high discrimination accuracy, which differs from each person, can be achieved. Further, as basing on DNA every one has, it can be made by standardized method. Furthermore, as combination of numbers of repeating times of STR is used, and as encoded process is also conducted, leakage of DNA information itself can be prevented, and then privacy can be protected.

### EFFECTS OF THE INVENTION

According to the additive to add discrimination means, which relates to the present invention, by analyzing arrangement of material making up composition matter and by applying the patterns of signs to each of the material, it becomes possible for a person who will conduct identification to get discrimination information. Namely, the composition matter itself of the additive becomes the discrimination means. Here, as it is impossible to obtain the discrimination information without depending on analyzing, there is no fear that the information is stolen easily by simple operation such as prior reading operation. Further, as it is impossible to make the discrimination information reappeared without making up composition matter by arranging specific materials according to the discrimination information, there is no fear that the information is forged easily. Moreover, as the discrimination means can be added by only adding, despite appearance of commodity needs to be verified, it is possible to form discrimination matter maintaining discrimination information which can be identified.

According to the matter maintaining discrimination information, which relates to the present invention, is to maintain the discrimination information indirectly through the discrimination means added by the additive. Then, as the discrimination information is that there is no fear of being stolen or forged easily, highly reliable discrimination can be achieved.

According to claim 2 and 7, as the discrimination information becomes digital, it is possible to use common information processing technology.

According to claim 3 and 8, as digitized discrimination information can be replaced by four kinds of materials, structure of the composition can be relatively simplified.

According to claim 4 and 9, to obtain a discrimination information, technology of DNA analyzing can be adapted.

According to claim 5, 10, 11 and 12, personal discrimination information with high discrimination accuracy, which differs from each person, can be achieved. Further, as basing on DNA every one has, it can be made by standardized method. Furthermore, as combination of numbers of repeating times of STR is used, and as encoded process is also conducted, leakage of DNA information itself can be prevented, and then privacy can be protected.

What is claimed is:

1. An additive to add discrimination means which characterized by consisting of composition matter (1) made up, according to a discrimination information (2) composed of more than one signs (21) repeatedly appear as more than one patterns (22), by making each of specific materials (11) be correspond to each of the patterns (22).

2. An additive to add discrimination means as claimed in claim 1, wherein said more than one signs (21) are 0 (21a) and 1 (21b).

3. An additive to add discrimination means as claimed in claim 1, wherein said more than one patterns (22) are 00 (22a), 01 (22b), 10 (22c) and 11 (22d).

4. An additive to add discrimination means as claimed in any one of claims 1 to 3, wherein said specific materials (11) are adenine (11a), cytosine (11b), guanine (11c) and thymine (11d), and said composition matter (1) is artificial DNA.

5. An additive to add discrimination means as claimed in any one of claims 1 to 3, wherein said discrimination information (2) is an information obtained by encoding combination (3) of numbers of repeating times of short tandem repeat (STR) in DNA base array.

6. A matter maintaining discrimination information which contains an additive consists of composition matter (1) made up, according to a discrimination information (2) composed of more than one signs (21) repeatedly appear as more than one patterns (22), by making each of specific materials (11) be correspond to each of the patterns (22).

7. A matter maintaining discrimination information as claimed in claim 6, wherein said more than one signs (21) are 0 (21a) and 1 (21b).

8. A matter maintaining discrimination information as claimed in claim 6, wherein said more than one patterns (22) are 00 (22a), 01 (22b), 10 (22c) and 11 (22d).

9. A matter maintaining discrimination information as claimed in any one of claims 6 to 8, wherein said specific materials (11) are adenine (11a), cytosine (11b), guanine (11c) and thymine (11d), and said composition matter (1) is artificial DNA.

10. A matter maintaining discrimination information as claimed in any one of claims 6 to 8, wherein said discrimination information (2) is an information obtained by encoding combination (3) of numbers of repeating times of short tandem repeat (STR) in DNA base array.

11. An additive to add discrimination means as claimed in claim 4, wherein said discrimination information (2) is an information obtained by encoding combination (3) of numbers of repeating times of short tandem repeat (STR) in DNA base array.

12. A matter maintaining discrimination information as claimed in claim 9, wherein said discrimination information (2) is an information obtained by encoding combination (3) of numbers of repeating times of short tandem repeat (STR) in DNA base array.
